(19) United States
(12) Patent Application Publication
(10) Pub. No.: US 2006/0141027 A1
(43) Pub. Date: Jun. 29, 2006

(54) Sublingual administration of non-steroidal anti-inflammatory pharmacological substances

(76) Inventor: Valerio Cioli, Roma (IT)

Correspondence Address: PEARNE & GORDON LLP, 1801 EAST 9TH STREET, SUITE 1200, CLEVELAND, OH, 44114-3108, US

(21) Appl. No.: 10/560,337

(22) PCT Filed: May 28, 2004
(86) PCT No.: PCT/IB04/01755

§ 371 Date: Dec. 9, 2005

(30) Foreign Application Priority Data
Jun. 10, 2003 (IT) RM2003A000288

(51) Int. Cl. A61K 9/20; A61K 31/192; A61K 31/165

(52) U.S. Cl. 424/464; 514/569; 514/570; 514/618; 514/563

(57) ABSTRACT

The present invention relates to a sublingual administration method of non-steroidal ant-inflammatory, referred as FANS, which allows to considerably reduce the therapeutic dose, with the additional advantage of increasing the quickness of the effects and improving the tolerability.

The present invention relates to a sublingual administration method of non-steroidal anti-inflammatories substances, referred as FANS hereinafter, which allows to considerably reduce its therapeutic dose, with the additional advantage of increasing the quickness of the effects and improving the tolerability.

FANS are drugs diffusely used for the control of inflammatory symptoms of different type, generally associated with pain and fever.

The oral administration, in the form of preparations to swallow, is the more common. It presents, however, some drawbacks which concern, in a more or less evident way, all this class of drugs.

Firstly, it is known that FANS may produce injury to the gastrointestinal system, consisting in ulcers, erosions and haemorrhages (Gabriel et al., 1991). This phenomenon is partly due to the central action mechanism of FANS, the same which also explains the anti-inflammatory properties thereof (Roberts and Morrow, 2001), partly to a contact action, which locally occurs on the gastrointestinal wall with which these drugs contact after being swallowed.

The damages of the first type declared themselves after the systemic absorption and are independent on the route of administration, while those of the second type precede the absorption and are bound to the oral administration.

Studies on animals and humans shown that the oral administration significantly contributes to the onset of these side effects. In rats, some FANS cause gastrointestinal lesions significantly more serious by oral way than after parenteral administration (Pfeiffer and Lewandowski, 1971; Cioli et al., 1979). The same phenomenon has been observed in humans and is explained in that, after oral administration, the FANS directly contact the gastrointestinal mucosa: in this way, their toxic local effects are added to those performed after the systemic absorption (Bjarnson and Thjodleifsson, 1999; Roberts and Morrow, 2001).

Another unfavourable aspect of the oral administration is to involve a first passage through the liver; consequently, FANS reach high concentrations in this organ, with a formation of reactive metabolites which can produce an oxidative stress and cause mitochondrial damages. In sensitive persons (metabolic idiosyncrasy), hepatotoxic reactions, serious as well, may result (Boesterli, 2002).

In order to reduce the drawbacks connected to the oral administration, FANS may be administered in gastro-resistent formulations, which do not deliver the active substance in the stomach. In this way, the gastric tolerability is improved but the erosions due to the direct contact of the active substance with the intestinal mucosa are not avoided, which can be equally dangerous (Davies, 1999). Further, FANS may be administered by injection and transdermically. In this way, the contact effect at the gastric and intestinal level may be avoided and the first passage through the liver is eliminated, thus being able to mitigate the hepatotoxic effects. Both these routes of administration, however, present drawbacks, which must be kept in mind (Wilkinson, 2001).

The injective route obliges to maintain the asepsis, may cause pain and makes the self-medication difficult.

The transdermic route is not always usable because of dosage problems and involves a slow absorption, hardly compatible with the treatment of acute inflammatory conditions, which need treatments promptly effective; few drugs, moreover, easily enter through the intact skin.

Finally, another possibility is offered by the rectal route, which however involves a rather irregular absorption and may irritate the mucosa of the last tract of the intestine; further, it reduces but it does not eliminate the effect of the first passage in the liver (Wilkinson, 2001).

Therefore, so far the sublingual administration of non-steroidal anti-inflammatory substances has never been subject of study or investigation.

Surprisingly, the above mentioned drawbacks are overcome through the sublingual administration of FANS, since from the oral cavity the drugs directly reach the superior vena cava, in this way the local component of the gastrointestinal damaging action is eliminate and the first massive passage through the liver is avoided.

The sublingual administration allows to considerably reduce the therapeutic dose, with respect to an oral formulation containing the same anti-inflammatory agent, with the advantage of increasing the quickness of the effects and ameliorating the tolerability. Further, the sublingual administration is easy to carry out.

The above mentioned advantages appeared by using, through sublingual administration, various active substances representative of the whole class of FANS, such as, for instance, nimesulide.

Nimesulide, as it is known, is particularly effective in the acute forms associated with pain. Its use, however, may cause adverse reactions to the gastrointestinal tract and, most of all, to the liver (REFI 2000).

In the performed experimentation, the used sublingual preparations consisted of tablets which can be separated in two parts.

During this experimentation, it unexpectedly emerged that the sublingual administration of FANS allows to remarkably reduce the therapeutic dose necessary for obtaining the desired anti-inflammatory effect.

The experimentation has further been closely examinated, both by treating in the following period the same patients with the traditional oral preparation and with the sublingual one, and by comparing groups of patients treated with the two methods. Besides allowing to considerably reduce the therapeutic dose, the sublingual administration presents the additional advantage of improving the quickness of the effects, which in the acute inflammatory conditions is of great importance, and the tolerability of FANS. The relative ascertainment to the dosage reduction required for obtaining a complete therapeutic effect was never been pointed out in the prior art.

Advantageously, the excipients used for the sublingual preparations of the tested FANS have been carefully selected among the available excipients in the pharmaceutical art.

The best excipients have proved to be those promoting the delivery of the active substance, by reducing the possible risk of local lesions for the oral mucosa which is exposed to the direct contact with the FANS. By using such types of excipients, during the experimentation carried out on patients, injuries of the type above mentioned have never been pointed out.

By way of example only, (Example 1), a tested preparation showed the following excipients composition:

| | | |
|---|---|---|
| Nimesulide | mg | 100.0 |
| Mannitol | mg | 200.0 |
| Sodium saccharate | mg | 30.0 |
| Microcrystalline cellulose | mg | 100.0 |
| PEG 6000 powder | mg | 5.0 |
| Citric acid | mg | 30.0 |
| Magnesium stearate | mg | 20.0 |
| Mint flavouring | mg | 20.0 |

By way of example only, (Example 2), another tested preparation showed the following excipients composition:

| | | |
|---|---|---|
| Nimesulide | mg | 50.0 |
| Mannitol | mg | 100.0 |
| Sodium saccharate | mg | 5.0 |
| Microcrystalline cellulose | mg | 50.0 |
| PEG 6000 powder | mg | 2.5 |
| Citric acid | mg | 25.0 |
| Magnesium stearate | mg | 5.0 |
| Mint flavouring | mg | 10.0 |

The patients seem to prefer the tablets of smaller sizes, but the reasons seem to be psychological only, as from the point of view of the therapeutic dosage reduction, the quickness of the effects and the tolerability, the size of the tablets and their formulations has not been influential. The reduction of the therapeutic dosage seems to depend, therefore, more from the route of administration than the formulation of the preparation, even if, of course, an influence of this latter cannot be excluded.

Greater advantages can be obtained by using tablets capable of quickly disintegrating, as the absorption of FANS is facilitated and the risk of local lesions is reduced.

All the sublingual preparations used in the experimentation are characterized by a prompt disintegration.

The experimentation has involved several FANS, such as, for example ketoprofen, nimesulide, naproxen and ibuprofen.

Moreover, other FANS provided with unusual physical-chemical features have been taken into consideration, such as, for instance, paracetamol, ketorolac, tenoxicam and diclofenac.

The present invention also applies to the 2-cyclo-oxigenase inhibitors, such as celecoxib and rophecoxib, with the advantage of a higher quickness of the therapeutic effects.

The experimentation carried out with the ketoprofen, the nimesulid and the ibuprofen on one hand, and with the paracetamol and diclofenac on the other hand, leads to consider that the observed reduction of the therapeutic dosage depends on the sublingual administration itself rather than the specific features of each drug.

A first group of patients subjected to the experimentation showed a clinical anamnesis of peptic ulcer or, in a more general sense, intolerance to the oral preparations of FANS. Afterward, the experimentation has been extended also to patients which well tolerated the traditional oral administration, by pointing out that also in those people the sublingual administration allows a drastic reduction of the therapeutic dose with respect to an oral formulation containing the same anti-inflammatory agent.

In order to more specifically show the invention, the following non limitative examples of galenical formulations are reported:

By way of example only, (Example 3), another experimented preparation showed the following composition:

| | | |
|---|---|---|
| Ketoprofen | mg | 25.0 |
| Mannitol | mg | 50.0 |
| Sodium saccharate | mg | 5.0 |
| Microcrystalline cellulose | mg | 25.0 |
| PEG 6000 powder | mg | 2.5 |
| Citric acid | mg | 12.5 |
| Magnesium stearate | mg | 3.0 |
| Mint flavouring | mg | 5.0 |

By way of example only, (Example 4), another experimented preparation showed the following composition:

| | | |
|---|---|---|
| Ibuprofen | mg | 100.0 |
| Mannitol | mg | 125.0 |
| Sodium saccharate | mg | 5.0 |
| Microcrystalline cellulose | mg | 75.0 |
| PEG 6000 powder | mg | 2.5 |
| Citric acid | mg | 25.0 |
| Magnesium stearate | mg | 5.0 |
| Mint flavouring | mg | 10.0 |

In general, the formulation according to the invention may be in a pharmaceutical form selected among: gel, granulate, powder, freeze-dried product, pressed capsule or pill.

Further, the pharmaceutical formulation according to the invention may include a water soluble excipient and/or a crystalline water insoluble excipient having a disintegrating function.

For instance, the water soluble excipient is the mannitol; the crystalline water insoluble excipient having a disintegrating function is the microcrystalline cellulose.

Moreover, the pharmaceutical formulation according to the invention may include: a lubricant, preferably said lubricant is the magnesium stearate and/or the PEG 6000 powder; a sweetener, preferably said sweetener is the sodium saccharate.

Of course, the common co-formulations usually used in the pharmaceutical technology may be employed without any limitation.

Finally, the formulations according to the invention are prepared according to the known teachings and the methods generally employed in the field.

### SOME MENTIONED REFERENCES ARE REPORTED HEREINAFTER

1. Bjarnason I., Thjodleifsson B.
   Gastrointestinal toxicity of non-steroidal anti-inflammatory drugs: the effect of nimesulide compared with naproxen on the human gastrointestinal tract, Rheumatology 1999 May, 38 Suppl 1: 24-32.
2. Boelsterli U. A.
   Mechanisms of NSAID-induced hepatotoxicity: focus on nimesulide, Drug Saf, 2002, 25 (9): 633-648.
3. Cioli V., Putzolu S., Rossi V., Scorza Barcellona P., Corradino C.
   The role of direct tissue contact in the production of gastrointestinal ulcers by anti-inflammatory drugs in rats, Toxicol. Appl. Pharmacol. 1979 Sep. 15; 50 (2): 283-289.
4. Gabriel S. E., Jaakkimainen L., Bombardier C.
   Risk for serious gastrointestinal complications related to use of nonsteroidal anti-inflammatory drugs. A meta-analysis, Ann Intern Med 1991 Nov 15, 115 (10): 787-796.
5. Davies N. M.
   Sustained release and enteric coated NSAIDs: are they really GI safe?, Pharm Pharm Sci 1999 January-April; 2 (1): 5-14.
6. Roberts II L. J., Morrow J. D.
   Analgesic-antipyretic and antiinflammatory agents and drugs employed in the treatment of gout, in Goodman and Gilman's, The pharmacological basis of therapeutics, 10th edition, eds. Hardman J. G. and Limbird L. E., 2001, McGraw-Hill, pag. 687-731.
7. Pfeiffer C. J., Lewandowski L. G.
   Comparison of gastric toxicity of acetylsalicylic acid with route of administration in the rat, Arch. Int. Pharmacodyn Ther. 1971 March; 190 (1): 5-13.
8. REFI (Repertorio Farmaceutico Italiano), 2000.
9. Wilkinson G. R.
   The Dynamics of drug absorption, distribution, and elimination, in Goodman and Gilman's, The pharmacological basis of therapeutics, 10th edition, eds. Hardman J. G. and Limbird L. E., 2001, McGraw-Hill, pag. 3-29.

1. Pharmaceutical formulation including a non-steroidal anti-inflammatory agent (FANS), characterized in that said formulation is administered by sublingual route and is such to promote the fast disintegration thereof in the oral cavity and is able to exert an anti-inflammatory activity at considerable reduced doses with respect to an oral formulation containing the same anti-inflammatory agent.

2. Formulation according to claim 1, characterized in that it contains a non-steroidal anti-inflammatory agent (FANS) capable of being absorbed by the oral mucosa; a water soluble excipient and/or a crystalline water insoluble excipient with a disintegrating function.

3. Formulation according to claim 2, characterized in that the water soluble excipient is mannitol.

4. Formulation according to claim 2, characterized in that the crystalline water insoluble excipient with a disintegrating function is microcrystalline cellulose.

5. Formulation according to claim 2, characterized in that it further contains a lubricant; preferably said lubricant is magnesium stearate and/or PEG 6000 powder.

6. Formulation according to claim 2, characterized in that it further contains a sweetener; preferably said sweetener is sodium saccharate.

7. Formulation according to at least one of the preceding claims, characterized in that the non-steroidal anti-inflammatory agent FANS is preferably selected among: nimesulide, ketoprofen, ibuprofen, paracetamol, diclofenac, naproxen, ketorolac, tenoxicam or pyroxicam.

8. Formulation according to at least one of the preceding claims, characterized in that said formulation is in a pharmaceutical form selected among: gel, granulate, powder, freeze-dried product, pressed capsule or pill.

9. Use of at least a non-steroidal anti-inflammatory agent FANS for the preparation of a sublingual pharmaceutical formulation for the treatment of inflammatory symptoms of various type, generally associated with pain and fever.

10. Use of at least a non-steroidal anti-inflammatory agent FANS for the preparation of a sublingual pharmaceutical formulation according to at least one of the claims 1-8 for the treatment of inflammatory symptoms of various type, generally associated with pain and fever.
